(19) United States
(12) Patent Application Publication
(10) Pub. No.: US 2007/0031886 A1
(43) Pub. Date: Feb. 8, 2007

(54) Detecting recessive diseases in inbred populations

(76) Inventor: Andrew A. Conway, Menlo Park, CA (US)

Correspondence Address: AGILENT TECHNOLOGIES INC., INTELLECTUAL PROPERTY ADMINISTRATION, M/S DU404, P.O. BOX 7599, LOVELAND, CO, 80537-0599, US

(21) Appl. No.: 11/581,132

(22) Filed: Oct. 13, 2006

Related U.S. Application Data
(63) Continuation of application No. 10/815,102, filed on Mar. 30, 2004.

(51) Int. Cl. C12Q 1/68; G06F 19/00

(52) U.S. Cl. 435/6; 702/20

(57) ABSTRACT

Techniques of using statistical analysis of genetic data to determine likely markers for a recessive genetic disease or trait. One embodiment of these techniques includes the steps of obtaining actual genotype data for one or more affected people with the genetic disease or trait in a population, obtaining estimated genotype data for the population, and analyzing the actual and estimated genotype data to find a region in genomes of the affected people that includes markers exhibiting particular homozygous pairs of alleles more frequently than would occur randomly.

### BACKGROUND OF THE INVENTION

1. Field of the Invention

The invention relates to detecting recessive diseases in inbred populations, such as for example moderately inbred populations such as the Amish population.

2. Background of the Invention

Many rare recessive diseases occur more frequently in certain inbred populations. One example of such a population is the Amish. Because the gene pool in an inbred population is more limited, expression of recessive genetic diseases can occur more frequently than in other populations. In particular, the chance can be higher for a child to inherit a matched pair of recessive alleles associated with a disease from his or her parents.

A brute-force approach could be used to try to correlate particular alleles with genetic diseases in the population. For example, it would be technically possible to sequence the entire genome of every member of one of these populations using conventional techniques. Gene sequences that coincide with occurrences of certain diseases could then be identified. However, extensive sequencing of an entire population, even a small one, would simply cost too much. Very few businesses and even governments would be able to afford the multi-billion dollar or even higher price for such an undertaking.

A more affordable technique would be to identify regions of the genome that are associated with the genetically-linked disease. Research can then focus on this region in a more cost effective manner.

### SUMMARY OF THE INVENTION

Accordingly, what is needed is a technique that tends to identify a general region of a human genome that contains genetic component(s) that contribute to or cause a genetically-linked recessive disease. The invention disclosed herein attempts to produce such results in the context of diseases that occur relatively more frequently in a relatively inbred population.

The invention addresses this need through techniques of using statistical analysis of genetic data to determine likely regions in the genome based upon markers there for a recessive genetic disease or trait. One embodiment of these techniques includes the steps of obtaining actual genotype data for one or more affected people with the genetic disease or trait in a population and/or actual genotype data for their parents, obtaining estimated genotype data for the population, and analyzing the actual and estimated genotype data to find a region in the genome of the affected people that includes markers exhibiting particular homozygous pairs of alleles more frequently than would occur randomly.

The techniques of the invention are particularly applicable to a population that is relatively inbred and that has a higher occurrence of the genetic disease or trait than a more general population. In such a population, the particular homozygous pairs of alleles that occur more frequently tend to be autozygous alleles descended from a founder of the genetic disease or trait.

In one embodiment, analyzing the genotype data further includes the steps of determining scores for each marker in the genotype data relative to each person for which actual genotype data was determined, merging the scores to arrive at a merged score for each marker, and determining a region of markers that has a high run of merged scores.

Preferably, a score for a marker represents a probability that a genotype measured for a person would actually be measured, given some assumption about the autozygosity at each marker's location. This approach results in a marker receiving a higher score from one form of homozygosity versus another form of homozygosity. The form that receives the higher score tends to be more likely to be associated with the genetic disease or trait.

After the scores are determined, they can be placed in an array ordered by a chromosomal order of markers associated with the scores. This facilitates analysis of the data, for example using a computer.

In one embodiment, the region of markers that has the high run of merged scores has the highest run of merged scores in the array. This region can be found by determining a consecutive portion of the array that has the highest sum. In this embodiment, runs of all possible lengths are considered. For example, if the total array of merged scores has 100 scores, the highest-scoring run might be 10 scores long, 20 scores long, or any other number of scores long.

High-scoring runs besides the highest-scoring run also can be of interest. For example, the next-highest runs might be of interest. Also, different techniques for finding runs of high scores (but not necessarily the highest run) can be used. In one such embodiment, the region of markers that has the high run of merged scores is found by computing all sums of a predetermined fixed number of adjacent elements in the array and comparing the sums. For example, if the total array of merged scores has 100 scores, the sums of all 10 score runs could be computed, resulting in 91 sums that could then be compared. Other techniques can be used.

Once a region with a high run of merged scores is found, traditional actual sequencing or other analysis can be performed on the DNA of the people in the population, preferably including people with the genetic disease or trait at issue, in or near the region that has the high run of merged scores. This sequencing hopefully will help find alleles or genetic patterns that cause the disease or trait. Because only this limited region is sequenced, this sequencing is far more affordable and feasible than sequencing the entire genome of every member of the subject population.

The invention also encompasses apparatuses, hardware, and software adapted to perform the steps of the foregoing techniques, as well as other embodiments of the invention.

After reading this application, those skilled in the art would recognize that the techniques described herein provide an enabling technology, with the effect that heretofore advantageous features can be provided that heretofore were substantially infeasible.

### BRIEF DESCRIPTION OF THE FIGURES

FIG. 1 illustrates inheritance of a genetic disease in a relatively inbred population.

FIG. 2 is an illustration of inheritance of alleles from parents to a child.

FIG. 3 is a flowchart showing steps for statistical analysis of genetic data according to one aspect of the invention.

FIG. 4 is a table showing calculations that can be used in the statistical analysis of genetic data.

FIG. 5 is a table showing results of calculations of scores for markers.

### DETAILED DESCRIPTION OF THE PREFERRED EMBODIMENT

Preferred embodiments of the invention are described herein, including preferred device coupling, device functionality, and process steps. After reading this application, those skilled in the art would realize that embodiments of the invention might be implemented using a variety of other techniques not specifically described herein, without undue experimentation or further invention, and that such other techniques would be within the scope and spirit of the invention.

Definitions

The general meaning of each of these terms is intended to be illustrative and in no way limiting.
The phrase “DNA” refers to a nucleic acid found in the nucleus of an organism's cells. DNA encodes information used by the organism to generate proteins, which in turn determine the physical characteristics of that organism. DNA is shaped from two strands connected together in a shape of a double helix.
The phrase “base pairs” refers to chemicals (i.e., nucleotides) that connect together the two strands that form a DNA double helix. The four possible base pair chemicals in DNA are adenine, thymine, guanine and cytosine. Adenine on one strand always bonds to thymine on the other strand in the double helix; guanine always bonds to cytosine. These chemicals are often abbreviated by their first letter (e.g., A, T, G and C).
The phrase “genome” refers to the entire DNA sequence of an organism such as a person. An organism's genome is often represented by a listing of abbreviations for the bases in the sequence, for example ATTACGGCACTG . . . .
The phrase “chromosome” refers to a portion of a human genome on which genetic sequences are linearly laid out; genetic sequences can be “near” each other on a chromosome if there are relatively few base pairs between them. Organisms include two copies of each chromosome, which are called homologues of each other. Each homologue of a chromosome includes the same markers, but can include different alleles for those markers.
The phrase “marker sequence” or “marker” refers to a genetic sequence (i.e., DNA found on a chromosome) that has more than one variant in the general population. Because an organism generally has to copies of each chromosome, the organism will have two copies of each marker, which may be the same or different from each other.
The phrase “allele” refers to any variant form of a marker. Alleles are often abbreviated with letters such as A, B, C, etc. The pair of alleles that a person has for the two copies of a particular marker is often abbreviated as AA, AB, BA, BB, AC, etc.
The phrase “genotype” refers to the particular genetic makeup at specified locations (e.g., markers) in the DNA of an organism.
The phrase “genotyping” refers to the process of determining a genotype for an organism.
The phrase “recessive” refers to a disease or trait that is only active if the same allele is present in both copies of the genetic variation that causes the disease or trait. The phrase “dominant” refers to a disease or trait that is active if even only one allele is present in both copies of the genetic variation that causes the disease or trait. For example, if A is an allele for a recessive disease or trait and B is an allele for a dominant disease or trait, a person with alleles AA generally will express the recessive disease or trait, while a person with alleles AB, BA, or BB generally will express the dominant disease or trait.
The phrase “homozygous” indicates two genetic sequences that are the same from both a person's mother and father. If homozygous genetic sequences are for an allele for a recessive genetic disease or trait, that disease or trait generally will be expressed in the person.
The phrase “heterozygous” indicates two genetic sequences that are different from the mother and the father.
The phrase “founder” refers to an individual, or a small set of individuals, who brought a disease sequence into a population.
The phrase “autozygous” indicates homozygous where the genetic sequences that are the same come from a common source such as a founder.
The phrase “disease sequence” refers to a genetic sequence, for example an allele, that causes or is associated with a particular disease.

The scope and spirit of the invention is not limited to any of these definitions, or to specific examples mentioned therein, but is intended to include the most general concepts embodied by these and other terms.

Overview

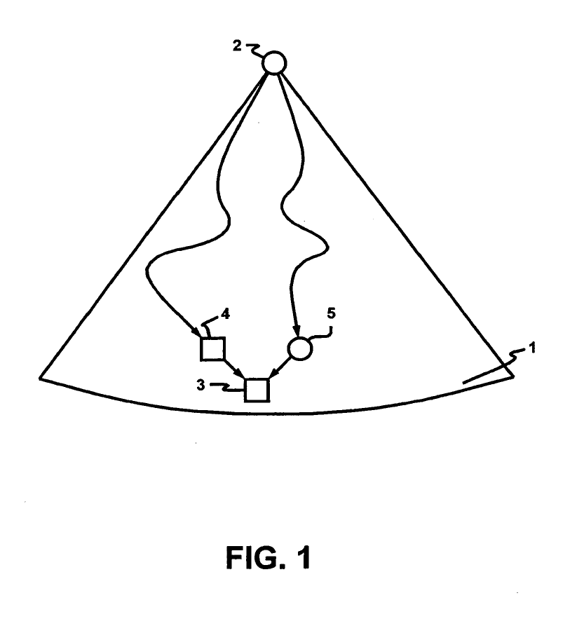

FIG. 1 illustrates inheritance of a genetic disease in a relatively inbred population.

In FIG. 1, population 1 is relatively inbred compared to a more general population. For example, the Amish population is relatively inbred compared to the general population of the U.S. or to the general population in regions where the Amish live.

At some time in the past, founder 2 introduced a genetic disease into the population. The disease is assumed to be recessive. Thus, in order for the disease to be expressed, a person must have two matching alleles for the disease at the corresponding location in the person's DNA.

In order to have two matching alleles for the disease, one must have come from the person's mother and one from the person's father. This situation is known as “homozygosity.” Furthermore, because these alleles were introduced by a single founder, the alleles are said to be “autozygous.”

In FIG. 1, founder 2 had at least two offspring that each carried one allele for the genetic disease introduced by the founder. These alleles were passed by subsequent offspring until they met at affected person 3 in the population through parents 4 and 5.

Generally, the paths taken by the alleles from a founder to an affected person do not cross. Otherwise, the person at whom they crossed would be an affected person. However, in some instances, the paths might cross. For example, if the disease is not terminal, the person might have passed one of the alleles on to a descendant. Likewise, if some other genetic or environmental factor is necessary for expression of the disease, the paths might have crossed without the disease being expressed.

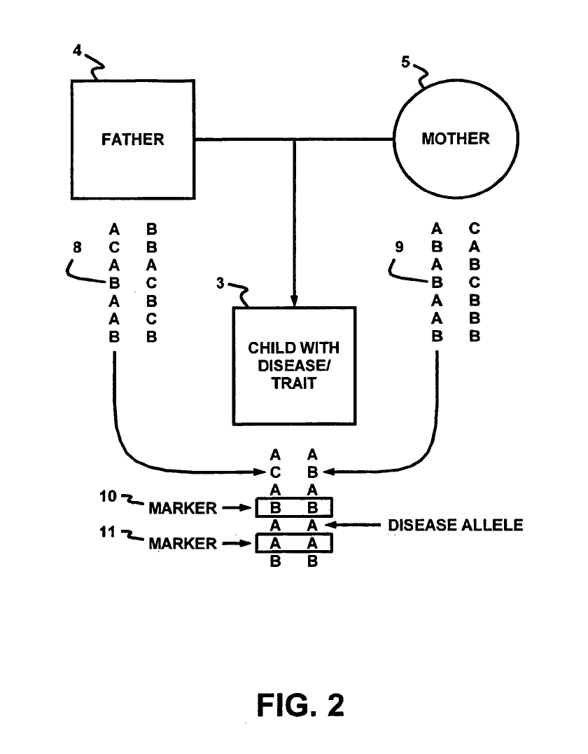

FIG. 2 is an illustration of inheritance of alleles from parents to a child. The particular combinations of alleles shown and discussed with respect to FIG. 2 are illustrative only. The invention is not limited to these particular alleles, markers, and disease alleles.

In FIG. 2, child 3 suffers from the recessive genetic disease under study. The child inherited one set of alleles 8 from father 4 and one set of alleles 9 from mother 5, as illustrated by the curved arrows.

The disease allele A is a recessive disease causing allele. Because two of these recessive alleles are present, the disease will be expressed in the child.

Marker alleles 10 and 11 are nearby alleles that are useful as markers. Father 4 and mother 5 in FIG. 2 each have one copy of these marker alleles.

In some cases, these alleles might be single nucleotide polymorphisms (SNPs). Other types of marker alleles can be used. For example, in FIG. 2, three different types of alleles are present, so these markers are not SNPs.

Both the disease alleles and the marker alleles are homozygous, meaning that they are the same from both the child's mother and father. The disease alleles and the nearby marker alleles ultimately originated with the founder (not shown). Thus, these alleles are also autozygous.

Alleles 8 and 9 are slightly different from each other because sets of alleles on a chromosome do not necessarily pass as a complete group. Some cross-over of alleles between homologues typically occurs from one generation to the next, resulting in mixing of alleles. The difference between alleles 8 and 9 (in the second marker from the top) could be the result of such cross-over at some point in the line of descent from the founder to the parents. Other causes (e.g., mutation) could also account for such differences, which may or may not be present to varying degrees.

One result of allele cross-over is that marker alleles from the founder might appear when disease alleles are not present, and marker alleles might be absent when disease alleles are present. However, nearby alleles are more likely to stay together from one generation to the next than distant alleles. Thus, the more common case is that the same nearby marker alleles appear in an, affected person as appeared in the founder.

Thus, FIG. 2 illustrates that a child with a pair of disease alleles is likely to have copies of nearby markers possessed by the founder. Furthermore, the parents are each likely to have at least one copy of the nearby markers.

The presence of these markers can be used to help locate a chromosomal region close to alleles causing or otherwise associated with the genetic disease. The overall approach of the invention is to try to find chromosomal regions for people with the disease under study that show a pattern more consistent than would occur by chance. Part of this pattern is the presence of homozygous alleles that occur more frequently than chance allows. Another part of this pattern is the presence of one type of homozygous alleles more frequently than other types.

In more detail, as discussed above, markers near to disease alleles tend to come from the same founder and tend to pass along with the disease alleles. As a result, the same pattern of marker alleles as found in the founder should tend to be more prevalent in affected people. Thus, in the example shown in FIG. 2, affect persons should have alleles BB for marker 10 and alleles AA for markers 11 much more frequently than other combinations of markers. Accordingly, particular combinations of homozygous markers that occur more frequently than other combinations of markers are of particular interest.

One embodiment of the invention that takes advantage of the foregoing observations is basically a two-step process.

First, scores are generated for each marker in the genotypes of members of a population that exhibit a recessive genetic disease. Each score represents a probability that a genotype measured for a person would actually be measured, given some assumption about the autozygosity at each marker's location.

Second, the scores are merged for all people in the population affected by the disease under consideration. This results in one score for each marker. Then, the scores are searched for a high or highest valued run. This run corresponds to markers that are likely to have descended along with the disease allele from the founder and therefore are likely to be close to the disease alleles.

Once a region likely to contain the disease allele is identified, actual sequencing of the DNA in or near this region can be performed using well known traditional techniques (or other techniques as they become developed). This sequencing can be performed on people with the genetic disease at issue, as well as on other people in the population. Because only a limited region of the DNA is being sequenced, this process is much more feasible than a brute-force sequencing of the entire genome (i.e., all the DNA) for every member of the population with the disease. Other known or developed techniques for studying the identified region also can be utilized

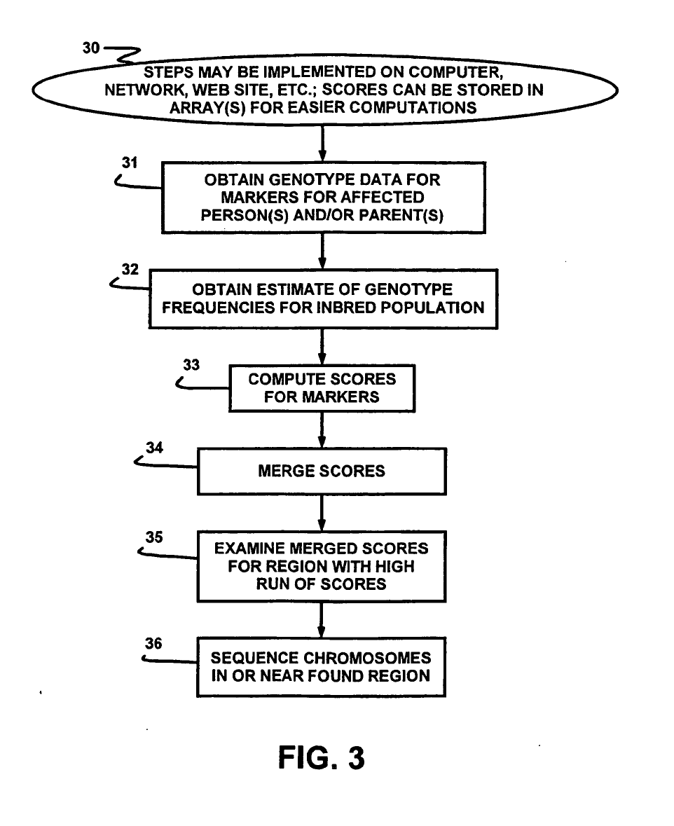

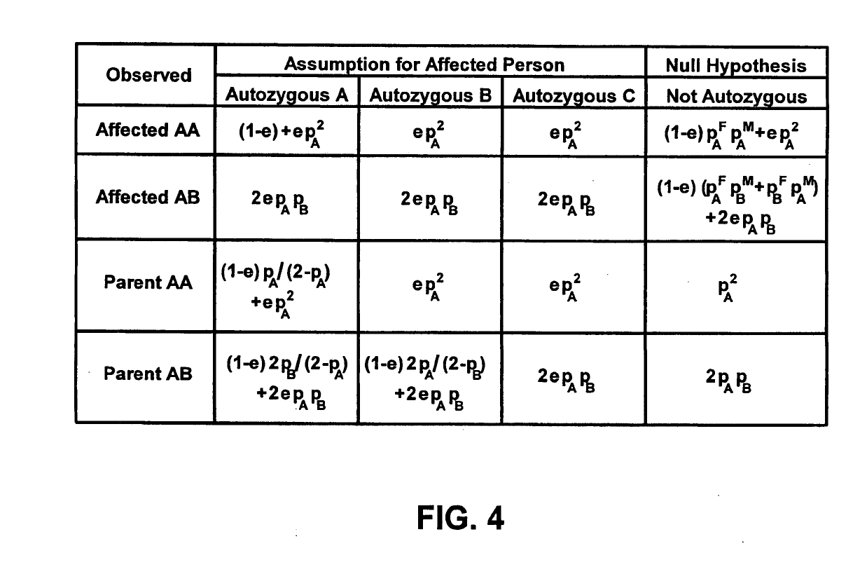

Steps for implementing the foregoing technique are discussed in more detail below with reference to FIGS. 3 and 4.

Statistical Analysis

FIG. 3 is a flowchart showing steps for statistical analysis of genetic data to determine likely markers for a recessive genetic disease or trait

As indicated in note 30, the steps in FIG. 3 can be implemented on a computer, network, web site, etc., using either general purpose or special purpose hardware and software. In these embodiments, arrays are particularly useful for handling genotype data and scores. Of course, the invention is not limited to use of arrays or to computer-implemented embodiments.

In step 31, actual genotype data is determined for one or more affected persons with the genetic disease under consideration. This genotype data is not a full sequencing of the person's DNA. Rather, the genotype data is an identification of particular alleles at a selected set of markers in the person's DNA. For example, a set of SNP markers could be determined for the affected person(s). Such genotyping is far less expensive than full DNA sequencing.

Actual genotype data also can be determined for the parents of affected persons.

In step 32, estimates are obtained of genotype frequency data for the entire inbred population to which the affected persons and their parents belong. When determining these estimates, it can be assumed that the alleles a child gets for any marker from his or her parents are independent.

In one embodiment, the estimates are found by actually genotyping a subset of the population. An error rate e for the estimates can be assumed, with the presence of the error indicating that a measured value in the genotyping is a result of a random selection from the population. Standard statistical techniques can be used to determine the error rate e from the size of the subset and the size of the overall population under consideration. Other techniques can be used to find the estimates without departing from the invention.

Scores are determined in step 33 for the markers selected for the genotyping. A score is determined in turn for each marker relative to each affected member or parent for which actual genotype data was determined in step 31.

FIG. 4 shows a table with probability calculations that can be used to determine the scores according to one embodiment of the invention. Several variables are used in these calculations, as follows:
n=a number of alleles possible for the marker under consideration, designated as A, B, C, etc.—for markers that are SNPs, n is usually two;
p_{X}=the estimated frequency of allele X in the population, as determined in step 32, with X being one of A, B, C, etc. (e.g., p_{A}=the estimated frequency of allele A at the marker, p_{B}=the estimated frequency of allele B at the marker, etc.);
p_{X}^{M}=the probability that an affected person got allele X at the marker under consideration from his or her mother—if the mother's genotype at the marker is known, this can be determined using standard Mendelian genetics and will be 0, 0.5, or 1; otherwise pₓ is used;
p_{X}^{F}=the probability that an affected person got allele X at the marker under consideration from his or her father—if the father's genotype at the marker is known, this can be determined using standard Mendelian genetics and will be 0, 0.5, or 1; otherwise p_{X} is used.

In order to find a score for a marker relative to an affected person or parent of an affected person, the row of the table in FIG. 4 is selected that corresponds to the observed genotype data for that person or parent. The calculations in that row are performed to determine probabilities of observing that marker given various types of autozygosity with the founder and also the probability of observing that marker in the absence of autozygosity.

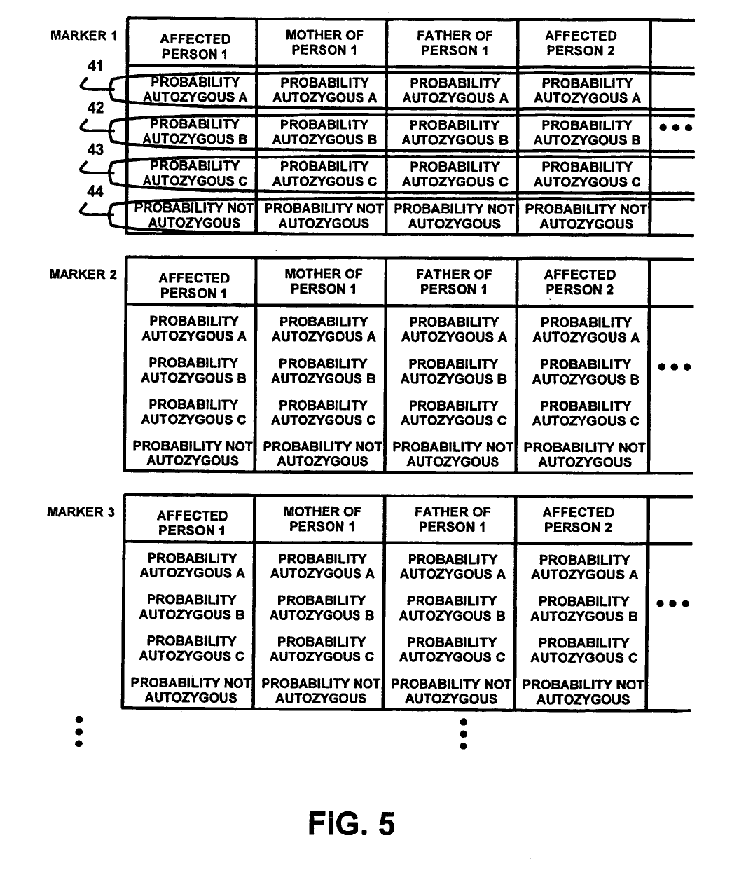

For each marker, this process is repeated relative to each affected person or parent of an affected person for whom actual genotype data is available. The result is a collection of scores for each marker representing probabilities of different types of autozygosity relative to each affected person or parent, as illustrated in FIG. 5.

Markers will receive higher scores for some forms of homozygosity as compared to other forms. The forms that receive the higher scores tend to be more likely to be associated with the genetic disease or trait.

The tables in FIGS. 4 and FIG. 5 can be expanded using basic rules of symmetry to accommodate other possible combinations of alleles. These tables can also be expanded to more complex pedigree information (i.e., grandparents).

Next, in step 34, the scores are merged.

First, scores for each type of autozygosity for each marker are multiplied together. For example, in FIG. 5, scores in group 41 are multiplied together, scores in group 42 are multiplied together, and scores in group 43 are multiplied together. This is repeated for all markers.

Second, the products for each type of autozygosity are summed weighted by the probability of that allele for that marker in the population. For example, the products from multiplying groups 41, 42 and 43 are summed. This is repeated for all markers. The result is a score representing the likelihood of observing the actual measured value for the marker given that the marker is autozygous (i.e., homozygous and inherited from the founder).

Third, scores for the “not autozygous” case for each marker are multiplied together. For example, scores in group 44 are multiplied together. This is repeated for all markers. The result is a score representing the likelihood of observing the actual measured value for the marker given that the marker is not autozygous and comes independently from the overall population distribution (i.e., is not from the founder).

More formally, if O is a set of genotype measurements believed to come from a single founder (i.e., genotypes of persons affected by the disease or trait under study), o is one of the genotypes in O, Pr(o|autozygous i) and Pr(o|not autozygous) come from the table in FIG. 5 (which in turn comes from the table in FIG. 4), and i is an index of different possible alleles at each marker, then

$$Pr ( O \text{\textbar} autozygous i ) = {\prod }_{o ∈ O} Pr ( o \text{\textbar} autozygous i ) , \\ Pr ( O \text{\textbar} autozygous ) = {\sum }_{i} {p}_{i} Pr ( O \text{\textbar} autozygous i ) , and$$

$$Pr ( O \text{\textbar} not autozygous ) = {\prod }_{o ∈ O} Pr ( o \text{\textbar} not autozygous ) .$$

Fourth, the ratio of Pr(O|autozygous) to Pr(O|not autozygous) is computed for each marker. Preferable, a log base 10 is taken of each ratio. More formally:

Marker Score=log₁₀[Pr(O|autozygous)/Pr(O|not autozygous)]

The resulting score is comparable to a LOD score obtained through different types of analysis such as genetic linkage or sib pair analysis.

The foregoing order of mathematical operations is chosen merely for the sake of convenience of explanation; other orders can be used without departing from the invention. These orders include, but are not limited to, maintaining running products and sums, performing simultaneous multiplication and summing operations, and the like.

The end result of step 34 is a score for each marker for which genotype data was collected. These scores can be arranged in an array or otherwise ordered in accordance with the order of the markers on chromosomes.

The scores themselves are intrinsically interesting because the computations up to this point are relatively conservative. Thus, high scores are very likely to be significant

In step 35, the merged scores are examined to find a run of high scores. In the preferred embodiment, the contiguous run of scores with the highest sum is found. Known techniques exist for finding a consecutive region with the highest sum in an array of numbers. One such technique is briefly described below:

1. Set a “running score” variable S to 0

2. Set a “current region start” variable C to clear

3. Set a “best region” variable B to clear

4. Set a “highest score” variable H to 0

5. Loop over all scores in the array in chromosomal order
a. Let MS be the Marker Score at the current place in the loop
b. Add MS to S
c. If S is zero or less, the marker is not interesting; set S to 0 and clear C
d. If S is greater than zero, the marker may be interesting; if C is clear, set C to this marker
   e. If S is greater than H, this is the best region so far; set B to start at C and end at this marker; set S to H

The chromosomal region corresponding to the “best region” B is likely to include or at least to be near the disease-causing alleles.

High-scoring runs besides the highest-scoring run also can be of interest. For example, the next-highest runs determined using the foregoing technique might be of interest A statistically significant jump or gap in scores between high-scoring runs and low-scoring runs could be used to select interesting regions. For example, if the highest scoring run has a score of 20, the next highest non-overlapping run has a score of 18 or 19, and the next nearest highest non-overlapping run has a score of 6, then the regions corresponding to scores of 18 or 19 and 20 might be of interest.

In addition, other techniques for finding runs of high scores (but not necessarily the highest run) can be used. In one such embodiment, the region of markers that has the high run of merged scores is found by computing all sums of a predetermined fixed number of adjacent elements in the array and comparing the sums. For example, if the total array of merged scores has 100 scores, the sums of all 10 score runs could be computed, resulting in 91 sums that could then be compared. Other techniques can be used.

Once a region with a high run of merged scores is found, actual sequencing of the DNA in or near this region can be performed in step 36 using well known traditional techniques (or other techniques as they become developed). This sequencing can be performed on people with the genetic disease at issue, as well as on other people in the population. Because only a limited region of the DNA is being sequenced, this process is much more feasible than a bruteforce sequencing of the entire genome (i.e., all the DNA) for every member of the population with the disease. Other known or developed techniques for studying the identified region also can be utilized

Genetic Traits Other than Disease

The foregoing discussion was in the context of a recessive genetic disease. However, the techniques of the invention are equally applicable to studies of recessive genetic traits. Application of these techniques to non-disease traits would not require further invention or undue experimentation.

Computer-Implemented Embodiments

Those skilled in the art would recognize, after perusal of this application, that embodiments of the invention may be implemented using one or more general purpose processors or special purpose processors adapted to particular process steps and data structures operating under program control, that such process steps and data structures can be embodied as information stored in or transmitted to and from memories (e.g., fixed memories such as DRAMs, SRAMs, hard disks, caches, etc., and removable memories such as floppy disks, CD-ROMs, data tapes, etc.) including instructions executable by such processors (e.g., object code that is directly executable, source code that is executable after compilation, code that is executable through interpretation, etc.), and that implementation of these process steps and data structures using such equipment would not require undue experimentation or further invention. For example, and without limitation, embodiments of the invention can be implemented on a desktop or laptop computer with standard input and output interfaces.

Alternative Embodiments

Although preferred embodiments are disclosed herein, many variations are possible which remain within the concept, scope, and spirit of the invention. These variations would become clear to those skilled in the art after perusal of this application.

After reading this application, those skilled in the art will recognize that these alterative embodiments and variations are illustrative and are intended to be in no way limiting.

After reading this application, those skilled in the art would recognize that the techniques described herein provide an enabling technology, with the effect that heretofore advantageous features can be provided that heretofore were substantially infeasible.

What is claimed is:

1. A method of using statistical analysis of genetic data to determine likely genetic regions for a recessive genetic disease or trait, comprising the steps of:
obtaining actual genotype data for one or more affected people with the genetic disease or trait in a population, for their parents, or for the affected people and their parents;
obtaining estimated genotype data for the population; and
analyzing the actual and estimated genotype data to find a region in genomes of the affected people that includes markers exhibiting particular homozygous pairs of alleles more frequently than would occur randomly, wherein the step of analyzing further comprises:
determining a set of scores under various assumptions for each marker in the genotype data relative to each person for which actual genotype data was determined;
merging the scores to arrive at a merged score for each marker; and
determining a region of markers that has a high run of merged scores.

2. A method as in claim 1, wherein the population is a relatively inbred population with a higher occurrence of the genetic disease or trait than a more general population.

3. A method as in claim 2, wherein the particular homozygous pairs of alleles are autozygous alleles descended from a founder of the genetic disease or trait in the relatively inbred population.

4. A method as in claim 3, wherein a score for a marker represents a comparison of a likelihood of observing the marker given that people with the genetic disease or trait are autozygous at the marker versus a likelihood of observing the marker given that alleles for the marker are independent of the genetic disease or trait.

5. A method as in claim 4, wherein a marker receives a higher score from one form of homozygosity versus another form of homozygosity, with the form receiving the higher score being more likely to be associated with the genetic disease or trait.

6. A method as in claim 5, wherein the merged scores are placed in an array ordered by a chromosomal order of markers associated with the scores.

7. A method as in claim 6, wherein the region of markers that has the high run of merged scores has the highest run of merged scores in the array; and
wherein the region of markers with the highest run of merged scores is found by determining a consecutive portion of the array that has the highest sum.

8. A method as in claim 6, wherein the region of markers that has the high run of merged scores is found by computing all sums of a predetermined fixed number of adjacent elements in the array and comparing the sums.

9. A method as in claim 6, further comprising the step of determining one or more additional regions of markers that have high runs of merged scores.

10. A method as in claim 9, further comprising the step of locating a statistically significant gap in the scores for non-overlapping regions, wherein regions having scores above the gap are determined to be the one or more additional regions of markers.

11. A method of analyzing actual and estimated genotype data, with the actual genotype data obtained for one or more affected people with the genetic disease or trait in a population, for their parents, or for the affected people and their parents, and with the estimated genotype data obtained for the population, the method performed to find a region in genomes of the affected people that includes markers exhibiting particular homozygous pairs of alleles more frequently than would occur randomly, the method comprising:
determining a set of scores under various assumptions for each marker in the genotype data relative to each person for which actual genotype data was determined;
merging the scores to arrive at a merged score for each marker; and
determining a region of markers that has a high run of merged scores.

12. A method as in claim 11, wherein the population is a relatively inbred population with a higher occurrence of the genetic disease or trait than a more general population.

13. A method as in claim 12, wherein the particular homozygous pairs of alleles are autozygous alleles descended from a founder of the genetic disease or trait in the relatively inbred population.

14. A method as in claim 13, wherein a score for a marker represents a comparison of a likelihood of observing the marker given that people with the genetic disease or trait are autozygous at the marker versus a likelihood of observing the marker given that alleles for the marker are independent of the genetic disease or trait.

15. A method as in claim 14, wherein a marker receives a higher score from one form of homozygosity versus another form of homozygosity, with the form receiving the higher score being more likely to be associated with the genetic disease or trait.

16. A method as in claim 15, wherein the merged scores are placed in an array ordered by a chromosomal order of markers associated with the scores.

17. A method as in claim 16, wherein the region of markers that has the high run of merged scores has the highest run of merged scores in the array; and
wherein the region of markers with the highest run of merged scores is found by determining a consecutive portion of the array that has the highest sum.

18. A method as in claim 16, wherein the region of markers that has the high run of merged scores is found by computing all sums of a predetermined fixed number of adjacent elements in the array and comparing the sums.

19. A method as in claim 16, further comprising the step of determining one or more additional regions of markers that have high runs of merged scores.

20. A method as in claim 19, further comprising the step of locating a statistically significant gap in the scores for non-overlapping regions, wherein regions having scores above the gap are determined to be the one or more additional regions of markers.

21. An apparatus including:
a processor,
input and output interfaces; and
a memory storing instructions executable by the processor to analyze actual and estimated genotype data, with the actual genotype data obtained for one or more affected people with the genetic disease or trait in a population, for their parents, or for the affected people and their parents, and with the estimated genotype data obtained for the population, the method performed to find a region in genomes of the affected people that includes markers exhibiting particular homozygous pairs of alleles more frequently than would occur randomly, the instructions including steps of: (a) determining a set of scores under various assumptions for each marker in the genotype data relative to each person for which actual genotype data was determined; (b) merging the scores to arrive at a merged score for each marker, and (c) determining a region of markers that has a high run of merged scores.
